SEQUENCE LISTING (91 sequences)

SEQ ID NO: 1 - protein, 8 aa, Artificial Sequence
  CDR1-H famille 1
GYTFTDYS

SEQ ID NO: 2 - protein, 8 aa, Artificial Sequence
  CDR2-H famille 1
  misc_feature (3)..(3): Xaa can be any naturally occurring amino acid
  misc_feature (8)..(8): Xaa can be any naturally occurring amino acid
ISXYYGDX

SEQ ID NO: 3 - protein, 11 aa, Artificial Sequence
  CDR3-H famille 1
  misc_feature (4)..(6): Xaa can be any naturally occurring amino acid
  misc_feature (8)..(10): Xaa can be any naturally occurring amino acid
ARNXXXYXXXY

SEQ ID NO: 4 - protein, 6 aa, Artificial Sequence
  CDR1-L famille 1
  misc_feature (4)..(4): Xaa can be any naturally occurring amino acid
QDIXNY

SEQ ID NO: 5 - skipped

SEQ ID NO: 6 - protein, 9 aa, Artificial Sequence
  CDR3-L famille 1
  misc_feature (4)..(4): Xaa can be any naturally occurring amino acid
QQGXTLPWT

SEQ ID NO: 7 - protein, 8 aa, Artificial Sequence
  CDR1-H famille 2
  misc_feature (7)..(7): Xaa can be any naturally occurring amino acid
GYTFTDXS

SEQ ID NO: 8 - protein, 8 aa, Artificial Sequence
  CDR2-H famille 2
  misc_feature (7)..(7): Xaa can be any naturally occurring amino acid
INTETGXP

SEQ ID NO: 9 - protein, 14 aa, Artificial Sequence
  CDR3-H famille 2
  misc_feature (1)..(1): Xaa can be any naturally occurring amino acid
  misc_feature (12)..(12): Xaa can be any naturally occurring amino acid
XRNGYYVGYYAXDY

SEQ ID NO: 10 - protein, 5 aa, Artificial Sequence
  CDR1-L famille 2
  misc_feature (4)..(4): Xaa can be any naturally occurring amino acid
SSVXY

SEQ ID NO: 11 - skipped

SEQ ID NO: 12 - protein, 9 aa, Artificial Sequence
  CDR3-L famille 2
  misc_feature (8)..(8): Xaa can be any naturally occurring amino acid
QQRRSYPXT

SEQ ID NO: 13 - protein, 8 aa, Artificial Sequence
  CDR1-H-IMGT-122A2
GYTFTDYS

SEQ ID NO: 14 - protein, 8 aa, Artificial Sequence
  CDR2-H-IMGT-122A2
ISTYYGDS

SEQ ID NO: 15 - protein, 11 aa, Artificial Sequence
  CDR3-H-IMGT-122A2
ARNGNFYVMDY

SEQ ID NO: 16 - protein, 6 aa, Artificial Sequence
  CDR1-L-IMGT-122A2
QDISNY

SEQ ID NO: 17 - skipped

SEQ ID NO: 18 - protein, 9 aa, Artificial Sequence
  CDR3-L-IMGT-122A2
QQGNTLPWT

SEQ ID NO: 19 - protein, 8 aa, Artificial Sequence
  CDR1-H-IMGT-102E9
GYTFTDYS

SEQ ID NO: 20 - protein, 8 aa, Artificial Sequence
  CDR2-H-IMGT-102E9
INTETGEP

SEQ ID NO: 21 - protein, 14 aa, Artificial Sequence
  CDR3-H-IMGT-102E9
TRNGYYVGYYAMDY

SEQ ID NO: 22 - protein, 5 aa, Artificial Sequence
  CDR1-L-IMGT-102E9
SSVIY

SEQ ID NO: 23 - skipped

SEQ ID NO: 24 - protein, 9 aa, Artificial Sequence
  CDR3-L-IMGT-102E9
QQRRSYPFT

SEQ ID NO: 25 - protein, 8 aa, Artificial Sequence
  CDR1-H-IMGT-104C12
GYTFTDYS

SEQ ID NO: 26 - protein, 8 aa, Artificial Sequence
  CDR2-H-IMGT-104C12
ISPYYGDT

SEQ ID NO: 27 - protein, 11 aa, Artificial Sequence
  CDR3-H-IMGT-104C12
ARNDDYYRFAY

SEQ ID NO: 28 - protein, 6 aa, Artificial Sequence
  CDR1-L-IMGT-104C12
QDINNY

SEQ ID NO: 29 - skipped

SEQ ID NO: 30 - protein, 9 aa, Artificial Sequence
  CDR3-L-IMGT-104C12
QQGKTLPWT

SEQ ID NO: 31 - protein, 8 aa, Artificial Sequence
  CDR1-H-IMGT-114D11
GYTFTDSS

SEQ ID NO: 32 - protein, 8 aa, Artificial Sequence
  CDR2-H-IMGT-114D11
INTETGGP

SEQ ID NO: 33 - protein, 14 aa, Artificial Sequence
  CDR3-H-IMGT-114D11
ARNGYYVGYYALDY

SEQ ID NO: 34 - protein, 5 aa, Artificial Sequence
  CDR1-L-IMGT-114D11
SSVFY

SEQ ID NO: 35 - skipped

SEQ ID NO: 36 - protein, 9 aa, Artificial Sequence
  CDR3-L-IMGT-114D11
QQRRSYPYT

SEQ ID NO: 37 - protein, 8 aa, Artificial Sequence
  CDR1-H-IMGT-104E10
GYTFTDYS

SEQ ID NO: 38 - protein, 8 aa, Artificial Sequence
  CDR2-H-IMGT-104E10
INTETGEP

SEQ ID NO: 39 - protein, 14 aa, Artificial Sequence
  CDR3-H-IMGT-104E10
ARNGYYVGYYAMDY

SEQ ID NO: 40 - protein, 5 aa, Artificial Sequence
  CDR1-L-IMGT-104E10
SSVIY

SEQ ID NO: 41 - skipped

SEQ ID NO: 42 - protein, 9 aa, Artificial Sequence
  CDR3-L-IMGT-104E10
QQRRSYPYT

SEQ ID NO: 43 - protein, 118 aa, Artificial Sequence
  VH-122A2
QVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSS

SEQ ID NO: 44 - protein, 121 aa, Artificial Sequence
  VH-102E9
QIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSS

SEQ ID NO: 45 - protein, 118 aa, Artificial Sequence
  VH-104C12
QVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSA

SEQ ID NO: 46 - protein, 121 aa, Artificial Sequence
  VH-114D11
QIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSS

SEQ ID NO: 47 - protein, 121 aa, Artificial Sequence
  VH-104E10
QIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSS

SEQ ID NO: 48 - protein, 107 aa, Artificial Sequence
  VL-122A2
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIK

SEQ ID NO: 49 - protein, 106 aa, Artificial Sequence
  VL-102E9
QIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIK

SEQ ID NO: 50 - protein, 107 aa, Artificial Sequence
  VL-104C12
DLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIR

SEQ ID NO: 51 - protein, 106 aa, Artificial Sequence
  VL-114D11
QIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIK

SEQ ID NO: 52 - protein, 106 aa, Artificial Sequence
  VL-104E10
QIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIK

SEQ ID NO: 53 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 107 aa, Homo sapiens [sequence omitted]

SEQ ID NO: 55 - protein, 447 aa, Artificial Sequence
  VH-CH-122A2
QVQLQQSGAELVRPGVSVKISCKGSGYTFTDYSMHWVKQSHAKSLEWIGVISTYYGDSNYNQKFKGKATMTVDKSSTTAYMELARLTSEDSAIYYCARNGNFYVMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 450 aa, Artificial Sequence
  VH-CH-102E9
QIHLVQSGPDLKKPGETVKISCKASGYTFTDYSMHWVKQAPGKGLKWMGWINTETGEPTYADDFKGRFAFSLESSASTAFLQINNLKNEDTSTYFCTRNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 447 aa, Artificial Sequence
  VH-CH-104C12
QVQLQQSGAELVGPGVSVKISCKGSGYTFTDYSMHWVKQSHAKSLEWIGVISPYYGDTNYNQKFKGKATMTVDKSSSTAYMELASLTSEDSAIYFCARNDDYYRFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 450 aa, Artificial Sequence
  VH-CH-114D11
QIQLVQSGPELKKPGETVKISCKASGYTFTDSSMHWVQQAPNKGLKWMGWINTETGGPTYADDFKGRFAFSLETSARTAYLQINNLKNEDTATYFCARNGYYVGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 450 aa, Artificial Sequence
  VH-CH-104E10
QIQLVQSGPELKKPGETVKISCKASGYTFTDYSMHWVKQAPGKGLKWMGWINTETGEPTYADDFKGRFAFSLETSATTAYLQINNFKNEDTATYFCARNGYYVGYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 214 aa, Artificial Sequence
  VL-CL-122A2
DIQMTQTTSSLSASLGDRVTISCRASQDISNYLNWYQQKPDGTVKLLIYYTSRLHSGVPSRFSGSGSGTDYSLTISNLDQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 213 aa, Artificial Sequence
  VL-CL-102E9
QIVLTQSPAIMSASPGEKVTITCSASSSVIYIHWFQQKPGTSPKLWIYSTSYLASGVPARFSGSGSGTSYSLTISRMEAEDAATYYCQQRRSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 214 aa, Artificial Sequence
  VL-CL-104C12
DLQMTQTPSSLSASLGDRVTISCRASQDINNYLSWYQEKPDGTFKLLIYYTSRLHSGVPSRFSGSGSGTDYSLTVRNLEQEDIGTYFCQQGKTLPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 213 aa, Artificial Sequence
  VL-CL-114D11
QIVLTQSPAIMSASPGEKVTITCSASSSVFYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 213 aa, Artificial Sequence
  VL-CL-104E10
QIVLTQSPAIMSASPGEKVTMTCSASSSVIYMHWFQQKPGTSPKLWIYSTSNLASGVPARFSGSGSGTSYSLTISRMEAEDAATYYCQQRRSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 106 aa, Artificial Sequence
  région variable de chaîne légère anti-AMHRII
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIK

SEQ ID NO: 66 - protein, 106 aa, Artificial Sequence
  région variable de chaîne légère anti-AMHRII
DIQMTQSPSTLSASVGDRVTITCRASSSVRYIAWYQQKPGKAPKLLIYSTSSLKSGVPSRFSGSGSGTEFTLTISSLQPDDFATYYCLQWSSYPWTFGGGTKVEIK

SEQ ID NO: 67 - protein, 106 aa, Artificial Sequence
  région variable de chaîne légère anti-AMHRII
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIK

SEQ ID NO: 68 - protein, 106 aa, Artificial Sequence
  région variable de chaîne légère anti-AMHRII
DIQMTQSPSTLSASVGDRVTITCRASSSVRYIAWYQQKPGKAPKLLIYPTSSLESGVPSRFSGSGSGTEFTLTISSLQPDDFATYYCLQWSSYPWTFGGGTKVEIK

SEQ ID NO: 69 - protein, 107 aa, Artificial Sequence [sequence omitted]

SEQ ID NO: 70 - protein, 115 aa, Artificial Sequence
  région variable de chaîne lourde anti-AMHRII
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 71 - protein, 115 aa, Artificial Sequence; identical to SEQ ID NO: 70
  région variable de chaîne lourde anti-AMHRII

SEQ ID NO: 72 - protein, 115 aa, Artificial Sequence
  région variable de chaîne lourde anti-AMHRII
QVQLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSS

SEQ ID NO: 73 - protein, 115 aa, Artificial Sequence
  région variable de chaîne lourde anti-AMHRII
QVQLVQSGAEVKKPGASVKVSCKASGYTFTSYHIHWVRQAPGQRPEWMGWIYPGDDSTKYSQKFQGRVTITRDTSASTAYMELSSLRSEDTAVYYCTRGDRFAYWGQGTLVTVSS

SEQ ID NO: 74 - protein, 115 aa, Artificial Sequence
  région variable de chaîne lourde anti-AMHRII
QVQLVQSGAEVKKPGASVKVSCKASGYTFTSYHIHWVRQAPGQRLEWMGWIYPGDDSTKYSQKFQGRVTITRDASASTAYMELSSLRSEDTAVYYCTRGDRFAYWGQGTLVTVSS

SEQ ID NO: 75 - protein, 330 aa, Artificial Sequence [sequence omitted]

SEQ ID NO: 76 - protein, 213 aa, Artificial Sequence
  VL 3C-23
DIQMTQSPSTLSASVGDRVTITCRASSSVRYIAWYQQKPGKAPKLLTYPTSSLESGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 445 aa, Artificial Sequence
  VH 3C-23
QVRLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 213 aa, Artificial Sequence
  VL 6b-78
DIQMTQSPSTLSASVGDRVTITCRASSSVRYIAWYQQKPGKAPKLLIYSTSSLKSGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 445 aa, Artificial Sequence
  VH 6b-78
QVQLVQSGAEVKKPGASVKVSCKASGYTFTSYHIHWVRQAPGQRLEWMGWIYPGDDSTKYSQKFQGRVTITRDTSASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 213 aa, Artificial Sequence
  VL 3c-23K
DIQMTQSPSTLSASVGDRVTITCRASSSVRYIAWYQQKPGKAPKLLTYPTSSLKSGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 445 aa, Artificial Sequence; identical to SEQ ID NO: 77
  VH 3c-23K

SEQ ID NO: 82 - protein, 213 aa, Artificial Sequence; identical to SEQ ID NO: 78
  VL 4C-35

SEQ ID NO: 83 - protein, 445 aa, Artificial Sequence
  VH 4C-35
QVQLVQSGAEVKKPGASVKVSCKASGYTFTSYHIHWVRQAPGQRPEWMGWIYPGDDSTKYSQKFQGRVTITRDTSASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 213 aa, Artificial Sequence
  VL 5B-42
DIQMTQSPSTLSASVGDRVTITCRASSSVRYIAWYQQKPGKAPKLLIYPTSSLESGVPSRFSGSGSGTEFTLTISSLQPDDFATYYCLQWSSYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - protein, 445 aa, Artificial Sequence
  VH 5B-42
QVQLVQSGAEVKKPGASVKVSCKASGYTFTSYHIHWVRQAPGQRLEWMGWIYPGDDSTKYSQKFQGRVTITRDASASTAYMELSSLRSEDTAVYYCTRGDRFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 213 aa, Homo sapiens
MVPEEEPQDREKGLWWFQLKVWSMAVVSILLLSVCFTVSSVVPHNFMYSKTVKRLSKLREYQQYHPSLTCVMEGKDIEDWSCCPTPWTSFQSSCYFISTGMQSWTKSQKNCSVMGADLVVINTREEQDFIIQNLKRNSSYFLGLSDPGGRRHWQWVDQTPYNENVTFWHSGEPNNLDERCAIINFRSSEEWGWNDIHCHVPQKSICKMKKIYI

SEQ ID NO: 87 - protein, 8 aa, Artificial Sequence
  CDR1-H 3C_23
GYTFTSHY

SEQ ID NO: 88 - protein, 8 aa, Artificial Sequence
  CDR2-H 3C_23
IYPGDDST

SEQ ID NO: 89 - protein, 8 aa, Artificial Sequence
  CDR3-H 3C_23
TRGDRFAY

SEQ ID NO: 90 - protein, 5 aa, Artificial Sequence
  CDR1-L 3C_23
SSVRY

SEQ ID NO: 91 - protein, 9 aa, Artificial Sequence
  CDR3-L 3C_23
LQWSSYPWT

(19) United States
(12) Patent Application Publication
(10) Pub. No.: US 2020/0095326 A1
(43) Pub. Date: Mar. 26, 2020

(54) COMBINATION OF ANT-CD303 AND ANTI-AMHRII ANTIBODIES

(71) Applicant: LABORATOIRE FRANCAIS DU FRACTIONNEMENT ET DES BIOTECHNOLOGIES, LES ULIS (FR)

(72) Inventors: Abdessatar Sami CHTOUROU, Elancourt (FR); Nathalie FOURNIER, Erquinghem-Lys (FR); Christophe DE ROMEUF, Lambersart (FR)

(21) Appl. No.: 16/469,173

(22) PCT Filed: Dec. 15, 2017
(86) PCT No.: PCT/EP2017/083156

§ 371 Date: Jun. 13, 2019

(30) Foreign Application Priority Data
Dec. 16, 2016 (FR) 16 62602

(51) Int. Cl. C07K 16/28; A61P 35/00

(52) U.S. Cl. CPC C07K 16/2851; C07K 16/2869; A61K 2039/507; C07K 2317/565; A61P 35/00

(57) ABSTRACT

Disclosed is a pharmaceutical composition including, in a pharmaceutically acceptable carrier: at least one anti-CD303 antibody; and at least one anti-AMHRII antibody. Also disclosed is the combination of the two aforementioned antibodies a) and b) to form a combination product to be used simultaneously, separately or spread out over a period of time, for the prevention or treatment of urogenital cancers. Also disclosed is a method for preventing or treating an urogenital cancer in a subject in need thereof, such method including administering to the subject products containing: at least one anti-CD303 antibody; and at least one anti-AMHRII antibody.

The subject of the present invention is a novel combination of antibodies, in particular for the prevention or treatment of urogenital cancers, preferably ovarian cancer.

Ovarian cancer is the main cause of gynaecological cancers and is the fifth cause of mortality in women through cancer; the three histological sites are the following:

surface epithelium (epithelial tumour with different sub-types) which represents 85-90% of ovarian cancers;

sex cords/stroma (granulosa cell tumour (3% of all ovarian cancers)) which represent about 10% of ovarian tumours; and

germ cells which represent 5% of ovarian cancers.

It is generally symptom-free in the early stages, giving it the nickname the « silent killer» (La Marca A., Volpe A. The Anti-Mullerian hormone and ovarian cancer. Human Reproduction Update, Vol.13, No.3 pp. 265-273, 2007).

According to FIGO classification (Fédération internationale de Gynécologie et d′Obstétrique), there are 4 stages at which survival rate strongly diminishes on and after stage 2:

Stage I: Tumour confined to the ovaries (five-year survival rate: 90-70%),

Stage II: Tumour in one or both ovaries with spreading to the pelvic region (five-year survival rate: 70-40%);

Stage III: Tumour in one or both ovaries with spreading beyond the pelvis (five-year survival rate: 20%); and

Stage IV: Metastasis to distant sites, with the exception of peritoneal metastases (five-year survival rate: <10%).

The chief strategies currently used to treat ovarian cancer are surgery or chemotherapy used in particular for first-line treatment e.g. a mixture of carboplatin and paclitaxel.

Monoclonal antibodies have also been recently developed such as cetuximab, which is directed against the epidermal growth factor receptor (EGFR, Ozols R. F. et al., Focus on epithelial ovarian cancer, Cancer Cell. 2004, Jan; 5(1): 19-24). Other immunotherapies are currently being researched such as those with abagovomab directed against CA-125, bevacizumab directed against the vascular endothelial growth factor (VEGF-A), farletuzumab, directed against folate receptor alpha (FRA), or mutated humanized anti-AMHRII antibodies (WO2011/141653). Targeted therapies e.g. with olaparib are additionally being tested.

However there still remains a need to optimise treatment of ovarian cancer, in particular when the cancer has invading effector cells likely to induce tolerance to the cancer by the immune system, whilst identifying a therapy that is better tolerated and has fewer side effects.

The inventors have now surprisingly discovered that the association of an anti-CD303 antibody and anti-AMHRII antibody is particularly effective against ovarian tumours, In particular, the combination of these two antibodies can exhibit a synergic effect at the site of the tumour.

The present invention therefore concerns a pharmaceutical composition comprising, in a pharmaceutically acceptable carrier:
a. at least one anti-CD303 antibody; and
b. at least one anti-AMHRII antibody.

The present invention also relates to products containing:
a. at least one anti-CD303 antibody; and
b. at least one anti-AMHRII antibody,

as combination products for simultaneous, separate or sequential use, for use in the prevention or treatment of urogenital cancers.

The invention further concerns:
at least one anti-AMHRII antibody for use in the prevention or treatment of urogenital cancers in combination with at least one anti-CD303 antibody; and
at least one anti-CD303 antibody for use in the prevention or treatment of urogenital cancers in combination with at least one anti-AMHRII antibody.

The invention also concerns a method for treating urogenital cancers, which comprises administering to a patient at least one anti-AMHRII antibody and at least one anti-CD303 antibody.

In the present invention, the term «antibody» refers both to an immunoglobulin and to fragments and derivatives of this immunoglobulin. Immunoglobulins are well-known to persons skilled in the art and are composed of an assembly of two dimers each formed of a heavy chain and a light chain. The multimeric complex is assembled by linking a light chain and a heavy chain via a disulfide bridge between two cysteines, the two heavy chains themselves also being linked together via two disulfide bridges.

Each of the heavy chains and light chains is composed of a constant region and a variable region. More specifically, each light chain is composed of a variable region (V_{L}) and a constant region (C_{L}). Each heavy chain is composed of a variable region (V_{H}) and a constant region formed of three constant domains CH₁, l CH₂ and CH_{3.} The domains CH₂ and C_{H3} form domain Fc. The variable regions of the light chain and of the heavy chain are composed of three regions determining recognition of the antigen (CDR regions i.e. Complementary Determining Regions) surrounded by four framework regions (FR regions).

The anti-CD303 a) and anti-AMHRII b) antibodies can be monoclonal or polyclonal. Preferably, they are monoclonal antibodies.

The antibodies can be of several isotypes, as a function of their type of constant region: the constant regions e γ, α, μ, ϵ and δ respectively correspond to immunoglobulins IgG, IgA, IgM, IgE and IgD. Advantageously, the anti-CD303 a) and anti-AMHRII b) antibodies are of IgG isotype. This isotype displays capability of efficiently inducing ADCC activity (« Antibody-Dependent Cellular Cytotoxicity) in the largest number of (human) individuals. The y constant regions comprises several subtypes: γ1, γ2, γ3, these three types of constant regions having the particularity of fixing human complement, and γ4, thereby creating the sub-isotypes IgG1, IgG2, IgG3 and IgG4. Advantageously, the anti-CD303 a) and anti-AMHRII b) antibodies are of IgG1 or IgG2 isotype, preferably IgG1.

In one particular aspect of the invention, the anti-CD303 a) and anti-AMHRII b) antibodies are selected from among murine antibodies, chimeric antibodies, humanized antibodies and human antibodies.

Preferably, the anti-CD303 a) antibody is a chimeric antibody, and more preferably a chimeric antibody selected from among a murine/human chimeric antibody or human/macaque chimeric antibody.

Alternatively, and preferably, the anti-CD303 a) antibody is a humanized antibody, and in particular a chimeric antibody in which the constant region of the heavy and light chains is of human origin.

The term «chimeric antibody» refers to an isolated antibody, in which its constituent sequence of each light chain and/or each heavy chain comprises or is composed of a hybrid sequence derived from at least two different animals. In particular, a chimeric antibody contains a light chain variable region and heavy chain variable region of murine wild-type, respectively fused with the light chain and heavy chain constant human regions. A chimeric antibody can be prepared using genetic recombination techniques well known to skilled persons.

The term «humanized antibody» refers to an antibody derived from an animal other than man and in which the sequences of the heavy chains and light chains other than CDRs have been replaced by corresponding sequences of one or more antibodies of human origin. The antibody is therefore mostly composed of human sequences, but the specificity thereof for the antigen imparted by the CDRs is derived from another species. In addition, some of the residues of backbone segments (called FRs) can be modified to maintain binding affinity (Jones et al.1986; Verhoeyen et al. 1988; Riechmann et al.1988). The humanized antibodies of the invention can be prepared using techniques known to skilled persons such as technologies of «CDR grafting», «resurfacing», SuperHumanisation, «Human string content», «FR libraries», «Guided selection», «FR shuffling» and «Humaneering», as summarized in the paper by Almagro et al. 2008.

As indicated above, the anti-CD303 and/or anti-AMHRII antibody of the invention can also be present in the form of a fragment of anti-CD303 antibody and/or fragment of anti-AMHRII antibody, respectively.

The term «fragment» refers in particular to the Fab, F(ab)′2 or Fd fragments.

The term «Fab» refers to an antibody fragment with a molecular weight of about 50 000 Da and having binding activity to the antigen. The Fab fragment is formed of the whole light chain (VL+CL) and part of the heavy chain (VH+CH1). It can be obtained in particular by treating IgGs with a protease: papain.

The term «F(ab′)2» refers to a fragment of about 100 000 Da and having binding activity to the antigen. It corresponds to the association via a disulfide bridge (hinge region) of two Fab fragments described above. It can be obtained by treating IgGs with a protease: pepsin.

The term «Fd» corresponds to that of the heavy chain included in the Fab fragment. The Fd fragment is therefore formed of the domains VH and CH1.

As indicated above, the anti-CD303 and/or anti-AMHRII antibody of the invention can also be present in the form of a derivative of an anti-CD303 a) antibody and/or derivative of an anti-AMHRII b) antibody, respectively.

The term «derivative» particularly refers to scFv derivatives, and to multimers of scFv e.g. the diabody, triabody or tetrabody.

The term «scFv» (single chain Fv) is a VH:VL polypeptide synthesised using the genes encoding the VL and VH domains and a linker sequence. A scFv includes the CDRs maintained in a suitable conformation e.g. using genetic recombination techniques.

scFvs can also be used as basic modules for the development of multimer structures (dimeric: «diabody»; trimeric: «triabody»; tetrameric: «tetrabody»).

The term «diabody » refers to a dimer of scFv. This dimeric fragment has the property of maintaining the dual valence of the parent antibody. The diabody is bivalent, mono- or bi-specific depending on whether it fixes two same or different antigens.

The term «triabody » refers to the trivalent association of scFvs. A triabody can therefore fix three same or different antigens.

The term «tetrabody » refers to the tetravalent association of scFvs. A tetrabody is able to fix four same or different antigens.

The term «urogenital cancers» refers to cancers of the urogenital tract in male or female organs. For example, it may concern prostate cancer, testicular cancer, penile cancer, endometrial cancer, vulvar or vaginal cancer, uterine cancer, cervical cancer, ovarian cancer, renal cancer, bladder cancer. Preferably, the urogenital cancer is ovarian cancer.

The present invention also concerns a pharmaceutical composition comprising, in a pharmaceutically acceptable carrier, at least one anti-CD303 antibody and at least one fragment or derivative of anti-AMHRII antibody.

The present invention also relates to products containing at least one anti-CD303 antibody, and at least one fragment or derivative of anti-AMHRII antibody, as combination products for simultaneous, separate or sequential use, for use in the prevention or treatment of urogenital cancers.

The present invention also concerns a pharmaceutical composition comprising, in a pharmaceutically acceptable carrier, at least one fragment of anti-CD303 antibody, and at least one anti-AMHRII antibody or one of the fragments thereof or one of the derivatives thereof.

The present invention also relates to products containing at least one fragment of anti-CD303 antibody, and at least one anti-AMHRII or one of the fragments thereof or one of the derivatives thereof, as combination products for simultaneous, separate or sequential use, for use in the prevention or treatment of urogenital cancers.

The present invention also concerns a pharmaceutical composition comprising, in a pharmaceutically acceptable carrier, at least one derivative of anti-CD303, antibody, and at least one anti-AMHRII antibody or one of the fragments thereof or one of the derivatives thereof.

The present invention also relates to products containing at least one derivative of anti-CD303 antibody, and at least one anti-AMHRII antibody or one of the fragments thereof or one of the derivatives thereof, as combination products for simultaneous, separate or sequential use, for use in the prevention or treatment of urogenital cancers.

The present invention also concerns a pharmaceutical composition comprising, in a pharmaceutically acceptable carrier, at least one anti-AMHRII antibody, and at least one fragment or derivative of anti-CD303 antibody.

The present invention also relates to products containing at least one anti-AMHRII antibody, and at least one fragment or derivative of anti-CD303 antibody, as combination products for simultaneous, separate or sequential use, for use in the prevention or treatment of urogenital cancers.

The present invention also concerns a pharmaceutical composition comprising, in a pharmaceutically acceptable carrier, at least one fragment of anti-AMHRII antibody, and at least one anti-CD303 antibody or one of the fragments thereof or one of the derivatives thereof.

The present invention also relates to products containing at least one fragment of anti-AMHRII antibody, and at least one anti-CD303 antibody or one of the fragments thereof or one of the derivatives thereof, as combination products for simultaneous, separate or sequential use, for use in the prevention or treatment of urogenital cancers.

The present invention also concerns a pharmaceutical composition comprising, in a pharmaceutically acceptable carrier, at least one derivative of anti-AMHRII antibody, and at least one anti-CD303 antibody or one of the fragments thereof or one of the derivatives thereof.

The present invention also relates to products containing at least one derivative of anti-AMHRII antibody, and at least one anti-CD303 antibody or one of the fragments thereof or one of the derivatives thereof, as combination products for simultaneous, separate or sequential use, for use in the prevention or treatment of urogenital cancers.

The present invention therefore concerns a pharmaceutical composition comprising, in a pharmaceutically acceptable carrier:
a. at least one anti-CD303 antibody; and
b. at least one anti-AMHRII antibody.

By «pharmaceutically acceptable carrier» is meant a non-toxic medium compatible with a biological system such as a cell, cell culture, tissue or organism.

The invention also relates to a product containing the two above-mentioned active agents a) and b), said active agents being combined for simultaneous, separate or sequential use, and in the prevention or treatment of urogenital cancers.

### Anti-CD303 Antibody

The anti-CD303 antibody is an antibody directed against the CD303 protein. This protein, also called BDCA-2, is specifically expressed on the surface of plasmacytoid dendritic cells, and it is a type II protein belonging to type-C lectins.

The human CD303 antigen (or CD303 protein) is the C member of lectin domain family 4 of type C (CLEC4 or «C-type lectin domain family 4, member C»). It is a type II transmembrane glycoprotein having 213 amino acids (accessible in Uniprot: Q8WTTO), comprising a short cytoplasmic domain without any evident signalling unit (amino acids 1-21), a transmembrane region (amino acids 22-44), and an extracellular domain (amino acids 45-213).

Plasmacytoid dendritic cells correspond to a sub-population of dendritic cells, also called DC2. Plasmacytoid dendritic cells are characterized by the Lin- markers (CD3−, CD19−, CD20−, CD14−, CD56−), HLA-DR+, CD11c−, CD123+and CD45RA+). These cells have also been phenotypically characterized: they express the markers CD4, CD303 and BDCA-4. They are present in lymphoid organs and also circulate in the blood. They are capable of secreting type-I IFN in the presence of a viral infection. They can promote the growth of tumour cells and survival thereof, in particular by inducing an immunosuppressive environment in the environment of the tumour e.g. by inducing differentiation of regulator T lymphocytes T (Treg). These cells therefore have immunosuppressive and/or tolerogenic properties towards a tumour. The expression «immunosuppressive properties» refers to the properties of dendritic cells to develop and maintain immunosuppression in the tumoral environment. The expression «tolerogenic properties» means that plasmacytoid dendritic cells will not induce an immune response.

The use of anti-CD303 antibodies of the invention, through their cytotoxic action, advantageously allows suppression of the plasmacytoid dendritic cells infiltrating the tumour. The immunosuppressive and/or tolerogenic properties towards the tumour are therefore reduced, advantageously removed, thereby improving anti-tumour immunity in situ.

Preferably, the anti-CD303 antibody of the invention is a monoclonal antibody directed against the ectodomain of the human CD303 antigen (SEQ ID NO :86).

Advantageously, the anti-CD303 antibody of the invention comprises heavy chains comprising three CDR-Hs (heavy chain CDRs according to IMGT nomenclature) having the following amino acid sequences, or sequences having at least 80% identity with the following sequences, and light chains comprising three CDR-Ls (light chain CDRs according to IMGT nomenclature) having the following amino acid sequences or sequences having at least 80% identity with the following sequences:

i) CDR1-H-family 1: SEQ ID NO: 1; CDR2-H-family 1: SEQ ID NO: 2; CDR3-H-family 1: SEQ ID NO: 3; CDR1-L-family 1: SEQ ID NO: 4; CDR2-L-family 1: SEQ ID NO: 5; CDR3-L-family 1: SEQ ID NO: 6; or

ii) CDR1-H-family 2: SEQ ID NO: 7; CDR2-H-family 2: SEQ ID NO: 8; CDR3-H-family 2: SEQ ID NO: 9; CDR1-L-family 2: SEQ ID NO: 10; CDR2-L-family 2: SEQ ID NO: 11; CDR3-L-family 2: SEQ ID NO: 12.

The expression « at least 80% identity >>means identity of 80%, 81%, 82%, 83%, 84%, 85%, 86%, 87%, 88%, 89%, 90%, 91%, 92%, 93%, 94%, 95%, 96%, 97%, 98%, 99% and 100%. The identity percentages to which reference is made in the present invention are determined on the basis of global alignment of the sequences to be compared i.e. on alignment of the sequences taken as a whole over their entire length using any algorithm known to skilled persons such as the Needleman-Wunsch algorithm-1970. This comparison of sequences can be carried out using any software known to skilled persons e.g. Needle software using a «Gap open» parameter of 10.0, «Gap extend» parameter of 0.5 and «Blosum 62»matrix. Needle software is available for example on the website ebi.ac.uk worldwide under the name «Align».

When the CDR or the variable region of an antibody has an amino acid sequence having at least 80%, preferably a least 85%, at least 90%, at least 91%, at least 92%, at least 93%, at least 94%, at least 95%, at least 96%, at least 97%, at least 98%, or at least 99% identity with a reference sequence, it may have insertions, deletions or substitutions compared with the reference sequence. If it has substitutions, substitution is preferably performed with an «equivalent» amino acid, i.e. any amino acid having a structure close to that of the original amino acid and is therefore not likely to modify the biological activities of the antibody, or an amino acid having a different structure but having intrinsic properties known to be equivalent to those of the original amino acid and not modifying the biological activities of the antibody.

Table 1 below summarises the amino acid sequences of the CDRs-IMGT of the two families of anti-CD303 antibodies able to be used in the invention:

TABLE 1
Amino acid sequences of the CDRs of the two
families of anti-CD303 antibodies able to be
used in the invention according to IMGT
nomenclature. In each sequence, X can represent
any amino acid.

| | Family 1 | Family 2 |
|---|---|---|
| CDR1-H | GYTFTDYS | GYTFTDXS |
| | (SEQ ID NO: 1) | (SEQ ID NO: 7) |
| CDR2-H | ISXYYGDX | INTETGXP |
| | (SEQ ID NO: 2) | (SEQ ID NO: 8) |
| CDR3-H | ARNXXXYXXXY | XRNGYYVGYYAXDY |
| | (SEQ ID NO: 3) | (SEQ ID NO: 9) |
| CDR1-L | QDIXNY | SSVXY |
| | (SEQ ID NO: 4) | (SEQ ID NO: 10) |
| CDR2-L | YTS | STS |
| | (SEQ ID NO: 5) | (SEQ ID NO: 11) |
| CDR3-L | QQGXTLPWT | QQRRSYPXT |
| | (SEQ ID NO: 6) | (SEQ ID NO: 12) |

Advantageously, the anti-CD303 antibody of the invention comprises three CDR-Hs (heavy chain CDRs according to IMGT nomenclature) having the following amino acid sequences, or sequences having at least 80% identity with the following sequences, and three CDR-Ls (light chain CDRs according to IMGT nomenclature) having the following amino acid sequences, or sequences having at least 80% identity with the following sequences:
i) CDR1-H-122A2: SEQ ID NO: 13, CDR2-H-122A2: SEQ ID NO: 14, CDR3-H-122A2: SEQ ID NO: 15, CDR1-L-122A2: SEQ ID NO: 16, CDR2-L-122A2: SEQ ID NO: 17, CDR3-L-122A2: SEQ ID NO: 18;
ii) CDR1-H-102E9: SEQ ID NO: 19, CDR2-H-102E9: SEQ ID NO: 20, CDR3-H-102E9: SEQ ID NO: 21, CDR1-L-102E9: SEQ ID NO: 22, CDR2-L-102E9: SEQ ID NO: 23, CDR3-L-102E9: SEQ ID NO: 24;
iii) CDR1-H-104C12: SEQ ID NO: 25, CDR2-H-104C12: SEQ ID NO: 26, CDR3-H-104C12: SEQ ID NO: 27, CDR1-L-104C12: SEQ ID NO: 28, CDR2-L-104C12: SEQ ID NO: 29, CDR3-L-104C12: SEQ ID NO: 30;
iv) CDR1-H-114D11: SEQ ID NO: 31, CDR2-H-114D11: SEQ ID NO: 32, CDR3-H-114D11: SEQ ID NO: 33, CDR1-L-114D11: SEQ ID NO: 34, CDR2-L-114D11: SEQ ID NO: 35, CDR3-L-114D11: SEQ ID NO: 36; or
v) CDR1-H-104E10: SEQ ID NO: 37, CDR2-H-104E10: SEQ ID NO: 38, CDR3-H-104E10: SEQ ID NO: 39, CDR1-L-104E10: SEQ ID NO: 40, CDR2-L-104E10: SEQ ID NO: 41, CDR3-L-104E10: SEQ ID NO: 42.

Advantageously, the anti-CD303 antibody of the invention has heavy and light chains with variable regions having the following amino acid sequences or sequences having at least 80% identity with the following sequences:
i) 122A2 antibody: heavy chain: SEQ ID NO: 43, light chain: SEQ ID NO: 48;
ii) 102E9 antibody: heavy chain: SEQ ID NO: 44, light chain: SEQ ID NO: 49;
iii) 104C12 antibody: heavy chain: SEQ ID NO: 45, light chain: SEQ ID NO: 50;
iv) 114D11 antibody: heavy chain: SEQ ID NO: 46, light chain: SEQ ID NO: 51; or
v)104E10 antibody: heavy chain: SEQ ID NO: 47, light chain: SEQ ID NO: 52.

Table 2 below summarises the amino acid sequences of the CDRs and of the variable regions of the heavy and light chains of the anti-CD303 antibodies of the invention:

TABLE 2
Amino acid sequences of CDR1s, CDR2s, and CDR3s
of the heavy and light chains according to IMGT
nomenclature, and of the VH and VL fragments of
the anti-CD303 antibodies of the invention.
122A2 antibody
Heavy chain

| | |
|---|---|
| CDR1-H- | GYTFTDYS |
| IMGT-122A2 | (SEQ ID NO: 13) |
| CDR2-H- | ISTYYGDS |
| IMGT-122A2 | (SEQ ID NO: 14) |
| CDR3-H- | ARNGNFYVMDY |
| IMGT-122A2 | (SEQ ID NO: 15) |
| VH-122A2 | QVQLQQSGAELVRPGVSVKISCKGSGYTFTDYSMHWVK |
| | QSHAKSLEWIGVISTYYGDSNYNQKFKGKATMTVDKSS |
| | TTAYMELARLTSEDSAIYYCARNGNFYVMDYWGQGTSV |
| | TVSS |
| | (SEQ ID NO: 43) |

Light chain

| | |
|---|---|
| CDR1-L- | QDISNY |
| IMGT-122A2 | (SEQ ID NO: 16) |
| CDR2-L-I | YTS |
| MGT-122A2 | (SEQ ID NO: 17) |
| CDR3-L- | QQGNTLPWT |
| IMGT-122A2 | (SEQ ID NO: 18) |
| VL-122A2 | DIQMTQTTSSLSASLGDRVTISCRASQDISNYLNWYQQ |
| | KPDGTVKLLIYYTSRLHSGVPSRFSGSGSGTDYSLTIS |
| | NLDQEDIATYFCQQGNTLPWTFGGGTKLEIK |
| | (SEQ ID NO: 48) |

102E9 antibody
Heavy chain

| | |
|---|---|
| CDR1-H- | GYTFTDYS |
| IMGT-102E9 | (SEQ ID NO: 19) |
| CDR2-H- | INTETGEP |
| IMGT-102E9 | (SEQ ID NO: 20) |
| CDR3-H- | TRNGYYVGYYAMDY |
| IMGT-102E9 | (SEQ ID NO: 21) |
| VH-102E9 | QIHLVQSGPDLKKPGETVKISCKASGYTFTDYSMHWVK |
| | QAPGKGLKWMGWINTETGEPTYADDFKGRFAFSLESSA |
| | STAFLQINNLKNEDTSTYFCTRNGYYVGYYAMDYWGQG |
| | TSVTVSS |
| | (SEQ ID NO: 44) |

Light chain

| | |
|---|---|
| CDR1-L- | SSVIY |
| IMGT-102E9 | (SEQ ID NO: 22) |
| CDR2-L- | STS |
| IMGT-102E9 | (SEQ ID NO: 23) |
| CDR3-L- | QQRRSYPFT |
| IMGT-102E9 | (SEQ ID NO: 24) |
| VL-102E9 | QIVLTQSPAIMSASPGEKVTITCSASSSVIYIHWFQQK |
| | PGTSPKLWIYSTSYLASGVPARFSGSGSGTSYSLTISR |
| | MEAEDAATYYCQQRRSYPFTFGGGTKLEIK |
| | (SEQ ID NO: 49) |

104C12 antibody
Heavy chain

| | |
|---|---|
| CDR1-H- | GYTFTDYS |
| IMGT-104C12 | (SEQ ID NO: 25) |
| CDR2-H- | ISPYYGDT |
| IMGT-104C12 | (SEQ ID NO: 26) |
| CDR3-H- | ARNDDYYRFAY |
| IMGT-104C12 | (SEQ ID NO: 27) |
| VH-104C12 | QVQLQQSGAELVGPGVSVKISCKGSGYTFTDYSMHWVK |
| | QSHAKSLEWIGVISPYYGDTNYNQKFKGKATMTVDKSS |
| | STAYMELASLTSEDSAIYFCARNDDYYRFAYWGQGTLV |
| | TVSA |
| | (SEQ ID NO: 45) |

Light chain

| | |
|---|---|
| CDR1-L- | QDINNY |
| IMGT-104C12 | (SEQ ID NO: 28) |
| CDR2-L- | YTS |
| IMGT-104C12 | (SEQ ID NO: 29) |
| CDR3-L- | QQGKTLPWT |
| IMGT-104C12 | (SEQ ID NO: 30) |
| VL-104C12 | DLQMTQTPSSLSASLGDRVTISCRASQDINNYLSWYQE |
| | KPDGTFKLLIYYTSRLHSGVPSRFSGSGSGTDYSLTVR |
| | NLEQEDIGTYFCQQGKTLPWTFGGGTKLEIR |
| | (SEQ ID NO: 50) |

114D11 antibody
Heavy chain

| | |
|---|---|
| CDR1-H- | GYTFTDSS |
| IMGT-114D11 | (SEQ ID NO: 31) |
| CDR2-H- | INTETGGP |
| IMGT-114D11 | (SEQ ID NO: 32) |
| CDR3-H- | ARNGYYVGYYALDY |
| IMGT-114D11 | (SEQ ID NO: 33) |
| VH-114D11 | QIQLVQSGPELKKPGETVKISCKASGYTFTDSSMHWVQ |
| | QAPNKGLKWMGWINTETGGPTYADDFKGRFAFSLETSA |
| | RTAYLQINNLKNEDTATYFCARNGYYVGYYALDYWGQG |
| | TSVTVSS |
| | (SEQ ID NO: 46) |

Light chain

| | |
|---|---|
| CDR1-L- | SSVFY |
| IMGT-114D11 | (SEQ ID NO: 34) |
| CDR2-L- | STS |
| IMGT-114D11 | (SEQ ID NO: 35) |
| CDR3-L- | QQRRSYPYT |
| IMGT-114D11 | (SEQ ID NO: 36) |
| VL-114D11 | QIVLTQSPAIMSASPGEKVTITCSASSSVFYMHWFQQK |
| | PGTSPKLWIYSTSNLASGVPARFSGSGSGTSYSLTISR |
| | MEAEDAATYYCQQRRSYPYTFGGGTKLEIK |
| | (SEQ ID NO: 51) |

104E10 antibody
Heavy chain

| | |
|---|---|
| CDR1-H- | GYTFTDYS |
| IMGT-104E10 | (SEQ ID NO: 37) |
| CDR2-H- | INTETGEP |
| IMGT-104E10 | (SEQ ID NO: 38) |
| CDR3-H- | ARNGYYVGYYAMDY |
| IMGT-104E10 | (SEQ ID NO: 39) |
| VH-104E10 | QIQLVQSGPELKKPGETVKISCKASGYTFTDYSMHWVK |
| | QAPGKGLKWMGWINTETGEPTYADDFKGRFAFSLETSA |
| | TTAYLQINNFKNEDTATYFCARNGYYVGYYAMDYWGQG |
| | TSVTVSS |
| | (SEQ ID NO: 47) |

Light chain

| | |
|---|---|
| CDR1-L- | SSVIY |
| IMGT-104E10 | (SEQ ID NO: 40) |
| CDR2-L- | STS |
| IMGT-104E10 | (SEQ ID NO: 41) |
| CDR3-L- | QQRRSYPYT |
| IMGT-104E10 | (SEQ ID NO: 42) |
| VL-104E10 | QIVLTQSPAIMSASPGEKVTMTCSASSSVIYMHWFQQK |
| | PGTSPKLWIYSTSNLASGVPARFSGSGSGTSYSLTISR |
| | MEAEDAATYYCQQRRSYPYTFGGGTKLEIK |
| | (SEQ ID NO: 52) |

Preferably, the anti-CD303 antibody of the invention has a human constant region, preferably a human constant region of IgG1 isotype.

The preferred constant region sequences of the human heavy or light chains, SEQ ID NO: 53 and SEQ ID NO: 54, of IgG1 isotype, are given in Table 3 below.

TABLE 3
Preferred sequences of human heavy and light
chain constant regions SEQ ID NO: 53 and
SEQ ID NO: 54.

| | |
|---|---|
| Preferred | ASTKGPSVFPLAPSSKSTSGGTAALGCLVKDYFPEPV |
| human | TVSWNSGALTSGVHTFPAVLQSSGLYSLSSVVTVPSS |
| heavy | SLGTQTYICNVNHKPSNTKVDKKVEPKSCDKTHTCPP |
| chain | CPAPELLGGPSVFLFPPKPKDTLMISRTPEVTCVVVD |
| constant | VSHEDPEVKFNWYVDGVEVHNAKTKPREEQYNSTYRV |
| region | VSVLTVLHQDWLNGKEYKCKVSNKALPAPIEKTISKA |
| (IgG1) | KGQPREPQVYTLPPSRDELTKNQVSLTCLVKGFYPSD |
| | IAVEWESNGQPENNYKTTPPVLDSDGSFFLYSKLTVD |
| | KSRWQQGNVFSCSVMHEALHNHYTQKSLSLSPG |
| | (SEQ ID NO: 53) |
| Preferred | RTVAAPSVFIFPPSDEQLKSGTASVVCLLNNFYPREA |
| human | KVQWKVDNALQSGNSQESVTEQDSKDSTYSLSSTLTL |
| light | SKADYEKHKVYACEVTHQGLSSPVTKSFNRGEC |
| chain | (SEQ ID NO: 54) |
| constant | |
| region | |
| (IgG1) | |

Therefore, the anti-CD303 antibody of the invention advantageously comprises the heavy and light chains described in Table 4 below.

TABLE 4
Amino acid sequences of the heavy and light chains
of the anti-CD303 antibodies of the invention.

| Antibody | Heavy chain | Light chain |
|---|---|---|
| 122A2 | Fusion SEQ ID NO: 43- | Fusion SEQ ID NO: 48- |
| | SEQ ID NO: 53 | SEQ ID NO: 54 |
| | (SEQ ID NO: 55) | (SEQ ID NO: 60) |
| 102E9 | Fusion SEQ ID NO: 44- | Fusion SEQ ID NO: 49- |
| | SEQ ID NO: 53 | SEQ ID NO: 54 |
| | (SEQ ID NO: 56) | (SEQ ID NO: 61) |
| 104C12 | Fusion SEQ ID NO: 45- | Fusion SEQ ID NO: 50- |
| | SEQ ID NO: 53 | SEQ ID NO: 54 |
| | (SEQ ID NO: 57) | (SEQ ID NO: 62) |
| 114D11 | Fusion SEQ ID NO: 46- | Fusion SEQ ID NO: 51- |
| | SEQ ID NO: 53 | SEQ ID NO: 54 |
| | (SEQ ID NO: 58) | (SEQ ID NO: 63) |
| 104E10 | Fusion SEQ ID NO: 47- | Fusion SEQ ID NO: 52- |
| | SEQ ID NO: 53 | SEQ ID NO: 54 |
| | (SEQ ID NO: 59) | (SEQ ID NO: 64) |

### Anti-AMHRII Antibody

The anti-AMHRII antibody is an antibody directed against the anti-Mullerian hormone receptor type II.

The human anti-Müllerian hormone (AMH) is a glycoprotein having 560 amino acids, and is a member of the TGF-β family. It is a hormone produced by the Sertoli cells of the foetal testis which causes degeneration of the Müllerian ducts. It is expressed in adults in the Sertoli and Leydig (testicle) cells and granulosa cells (ovary). It plays a role in the activity of adult ovaries by regulating folliculogenesis. The sequence of human AMH is accessible in Uniprot under reference P03971.

The anti-Müllerian hormone receptor type II (AMHR-II) is a peptide of 573 amino acids and has serine-threonine kinase activity. It is involved in regression of the Müllerian ducts associated with development of the male reproductive system. It becomes atrophied in males in whom it only forms the prostatic utricle and hydatid of Morgagni, but persists in females developing into the fallopian tubes, uterus and most of the vagina. This receptor is frequently expressed on epithelial tumour cells of human ovaries and granulosa tumours. The sequence f human AMHRII is accessible in Uniprot under reference Q16671.

Preferably, the anti-AMHRII antibody of the invention derives from the monoclonal murine antibody 12G4, described in particular in application WO2008/053330.

Preferably, the anti-AMHRII antibody of the invention is a humanized and mutated antibody, or fragments and derivatives thereof, having affinity at least equal to that of the corresponding non-mutated chimeric antibody, having specificity for the AMHRII receptor, and not triggering an immune reaction.

In one advantageous embodiment, the present invention concerns a mutated, humanized monoclonal 12G4 antibody such as defined above, having a light chain and heavy chain selected from among the following:

Preferably the anti-AMHRII antibody of the invention has:
a) a light chain comprising or composed of a variable region in which the amino acid sequence is represented by:
SEQ ID NO: 65, or
SEQ ID NO: 66, or
SEQ ID NO: 67, or
SEQ ID NO: 68,
and of a constant region in which the amino acid sequence is represented by SEQ ID NO: 69;
b) a heavy chain comprising or composed of a variable region in which the amino acid sequence is represented by:
   SEQ ID NO: 70,
   SEQ ID NO: 71,
   SEQ ID NO: 72,
   SEQ ID NO: 73,
   SEQ ID NO: 74,
and of a constant region in which the amino acid sequence is represented by SEQ ID NO: 75.

In one advantageous embodiment, the anti-AMHRII antibody of the invention has:
a) a light chain composed of the amino acid sequence represented by:
   SEQ ID NO: 76, and
b) a heavy chain composed of the amino acid sequence represented by:
   SEQ ID NO: 77,
   (3C_23 antibody);
or:
a) a light chain composed of the amino acid sequence represented by:
   SEQ ID NO: 78, and
b) a heavy chain composed of the amino acid sequence represented by:
   SEQ ID NO: 79,
(6B_78 antibody);
or:
a) a light chain composed of the amino acid sequence represented by:
   SEQ ID NO: 80, and
b) a heavy chain composed of the amino acid sequence represented by:
   SEQ ID NO: 81,
(3C_23K antibody);
or:
a) a light chain composed of the amino acid sequence represented by:
   SEQ ID NO: 82, and
b) a heavy chain composed of the amino acid sequence represented by:
   SEQ ID NO: 83,
   (4C_35 antibody);
or:
a) a light chain composed of the amino acid sequence represented by:
   SEQ ID NO: 84, and
b) a heavy chain composed of the amino acid sequence represented by:
   SEQ ID NO: 85,
(5B_42 antibody).

Preferably, the anti-AMHRII antibody of the invention comprises three CDR-Hs (heavy chain CDRs according to IMGT nomenclature) having the following amino acid sequences, or sequences having at least 80% identity with the following sequences, and three CDR-Ls (light chain CDRs according to IMGT nomenclature) having the following amino acid sequences or sequences having at least 80% identity with the following sequences:

| | | |
|---|---|---|
| CDR1-H 3C_23: | GYTFTSHY, | (SEQ ID NO: 87) |
| CDR2-H 3C_23: | IYPGDDST, | (SEQ ID NO: 88) |
| CDR3-H 3C_23: | TRGDRFAY, | (SEQ ID NO: 89) |
| CDR1-L 3C_23: | SSVRY, | (SEQ ID NO: 90) |
| CDR2-L 3C_23: | PTS | |
| and | | |
| CDR3-L 3C_23: | LQWSSYPWT. | (SEQ ID NO: 91) |

These 6 CDRs are those present in the 3C_23 antibody and 3C_23K antibody.

Preferably, the anti-AMHRII antibody of the invention comprises:
a) a light chain composed of the amino acid sequence represented by SEQ ID NO: 76, and
b) a heavy chain composed of the amino acid sequence represented by SEQ ID NO: 77, (3C_23 antibody);
or:
a) a light chain composed of the amino acid sequence represented by SEQ ID NO:

80, and
b) a heavy chain composed of the amino acid sequence represented by SEQ ID NO: 81, (3C_23K antibody).

The anti-CD303 and anti-AMHRII antibodies of the invention can each independently be produced in a host cell, a transgenic non-human animal or transgenic plant comprising at least one nucleic acid coding for said antibody, the fragments or derivatives thereof, or a vector containing said nucleic acid.

Preferably, the anti-CD303 antibody and/or anti-AMHRII antibody of the invention are produced via transgenesis, in particular in a non-human transgenic animal or transgenic plant.

The host cell can be of prokaryote or eukaryote origin, and selected in particular from among bacterial cells, insect, plant, yeast or mammalian cells. The antibody of the invention can then be produced by culture of the host cell under suitable conditions. A host cell of the invention can notably be obtained by transforming a cell line with the expression vector(s) of the heavy and light chains of an antibody of the invention, and by separating the different cell clones obtained. The transformed cell line is preferably of eukaryote origin and can be selected in particular from among insect, plant, yeast or mammalian cells. Suitable cell lines to produce antibodies notably include the lines selected from among: SP2/0; YB2/0; IR983F; Namalwa human myeloma; PERC6; CHO lines in particular CHO-K-1, CHO-Lec10, CHO-Lec1, CHO-Lec13, CHO Pro-5, CHO dhfr-, or CHO line with deletion of the two alleles encoding the FUT8 gene and/or GMD gene; Wil-2; Jurkat; Vero; Molt-4; COS-7; 293-HEK; BHK; K6H6; NSO; SP2/0-Ag 14, P3X63Ag8.653, duck embryo cell line EB66® (Valneva); rat hepatoma lines H4-II-E (DSM ACC3129) and H4-II-Es (DSM ACC3130) (see W02012/041768), NM-H9D8 (DSM ACC2806), NM-H9D8-E6 (DSM ACC 2807) and NM H9D8-E6Q12 (DSM ACC 2856) (see WO2008/028686).

A non-human transgenic animal of the invention can be obtained by direct injection of the gene(s) of interest into a fertilized egg (Gordon et al. 980). A non-human transgenic animal can also be obtained by inserting the gene(s) of interest into an embryonic stem cell and preparing the animal by chimera aggregation or chimera injection method (see Manipulating the Mouse Embryo, A Laboratory Manual, Second Edition, Cold Spring Harbor Laboratory Press (1994); Gene Targeting, A Practical Approach, IRL Press at Oxford University Press (1993)). A non-human transgenic animal can also be obtained using a cloning technique wherein a nucleus in which the gene(s) of interest have been inserted is transplanted into an enucleated egg (Ryan et al.1997; Cibelli et al.1998, WO00/26357). A non-human transgenic animal producing an antibody of interest can be prepared with the above methods. The antibody can then be accumulated in the transgenic animal and purified, in particular from the milk or eggs of the animal. For the production of antibodies in the milk of non-human transgenic animals, preparation methods are notably described in WO90/04036, WO95/17085, WO01/26455, WO2004/050847, WO2005/033281, WO2007/048077. Methods for purifying proteins of interest from the milk are also known (see WO01/26455, WO2007/106078). The non-human transgenic animals of interest particularly include mouse, rabbit, rat, goat, bovines (cow in particular) and poultry (chicken in particular).

A transgenic plant of the invention can be selected from among any plant allowing the production of antibodies. Numerous antibodies have already been produced in transgenic plants and the technologies required for obtaining a transgenic plant expressing an antibody of interest and for recovering the antibody are well known to skilled persons (see Stoger et al. 2002, Fisher et al. 2003, Ma et al. 2003, Schillberg et al. 2005). It is also possible to influence the glycosylation obtained in the plants, to obtain glycosylation close to that of the natural human antibodies (xylose-free), but additionally with low fucosylation e.g. by means of small interfering RNAs (Forthal et al. 2010).

The anti-CD303 antibody and anti-AMHRII antibody are present, according to the invention, either in a pharmaceutical composition or as combination products.

They can therefore be combined with pharmaceutically acceptable excipients and optionally with extended-release matrixes such as biodegradable polymers.

The pharmaceutical composition or combination product can be administered via oral, sublingual, subcutaneous, intramuscular, intravenous, intra-arterial, intrathecal, intra-ocular, intra-cerebral, transdermal, pulmonary, local or rectal route. The antibodies can be then be administered in unit administration form in a mixture with conventional pharmaceutical carriers. Unit administration forms comprise forms via oral route such as tablets, capsules, powders, granules and oral solutions or suspensions, sublingual and buccal administration forms, aerosols, subcutaneous, transdermal, topical, intraperitoneal, intramuscular, intravenous, subcutaneous, intrathecal implants, administration forms via intranasal route and rectal administration forms.

Preferably, the pharmaceutical composition or combination product contains a pharmaceutically acceptable carrier for a formulation able to be injected. In particular, these may be isotonic or sterile formulas, saline solutions or freeze-dried compositions which, when sterilized water or physiological saline solution accordingly are added thereto, allow the preparation of injectable solutions.

Suitable pharmaceutical forms for injectable use comprise sterile aqueous solutions or dispersions, oily formulations, and sterile powders for extemporaneous preparation of sterile injectable solutions or dispersions. In all cases, the form must be sterile and fluid insofar as it must be injected through a syringe. It must be stable under production and storage conditions, and must be protected against contaminating action of microorganisms such as bacteria and fungi.

The dispersions of the invention can be prepared in glycerol, liquid polyethylene glycols or mixtures thereof, or in oils. Under normal conditions of storage and use, these preparations contain a preserving agent to prevent the growth of micro-organisms.

The pharmaceutically acceptable carrier can be a solvent or dispersion medium. Suitable fluidity can be maintained for example by using a surfactant. Prevention of action by microorganisms can be obtained via various antibacterial and antifungal agents. In many cases, it will be preferable to include isotonic agents. Extended absorption of the injectable compositions can be obtained through the use of absorption-delaying agents.

Sterile injectable solutions are prepared by incorporating the active substances in required amount in a suitable solvent with some of the other ingredients listed above, optionally followed by filter sterilization. As a general rule, the dispersions are prepared by incorporating the various active sterilized ingredients in a sterile carrier containing the basic dispersion medium and the other ingredients required from among those listed above. With regard to sterile powders, to prepare sterile injectable solutions, the preferred preparation methods are vacuum drying and freeze-drying. At the time of formulation, the solutions are to be administered in a manner compatible with the dosage formulation in a therapeutically effective amount. The formulations are easily administered in a variety of pharmaceutical forms, such as the injectable solutions described above, but drug release capsules and the like can also be used. For parenteral administration in an aqueous solution for example, the solution must be suitably buffered and the liquid diluent made isotonic with sufficient saline solution or glucose. These particular aqueous solutions are particularly suitable for intravenous, intramuscular, subcutaneous and intraperitoneal administration. In this respect, the sterile aqueous media able to be used are known to those skilled in the art.

The level of therapeutically effective dose specific for a particular patient will depend on a variety of factors, including the disorder being treated and the seriousness of the disease, the activity of the specific compound employed, the specific composition used, patient age, bodyweight, general state of health, gender and diet, the time of administration, route of administration, the excretion rate of the specific compound used, the length of treatment or medications used in parallel.

The invention will now be illustrated with the following examples.

### EXAMPLES

### Example 1: Impact of pDC Depletion on ADCC and Phagocytosis Activities of Anti-AMHRII Antibodies

### 1.1-Principle of the Study

are responsible for the differentiation of regulatory T cells (Moseman E A, 2004; Martin-Gayo E, 2010) inter alia via ICOS/ICOSL interaction (Ito, T., 2007; Faget, J. 2012; Faget, J. 2013). The regulatory T cells thus differentiated exert immunosuppressive mechanisms on the functions of the other cells in the immune system, NK cells in particular, via cell/cell contact but also via the secretion of immuno-modulating cytokines such as IL-10, IL-35 and TGF-13 (Liu, C 2016).

Through cascade effect, it is therefore possible to examine the impact of pDC depletion on the action of an anticancer agent specific to a tumour involving in situ activation of pDCs. In particular, the protective effect of anti-CD303 antibodies targeting pDCs can be shown in a correlative study on the reduction or removal of IL-10 and TGF-β cytokines in the tumour environment, and the impact thereof on the effector functions of an administered anticancer agent (anti-AMHRII antibody) specific to the tumour. According to this model, the anti-CD303 antibodies depleting the pDCs lead to limiting the immunosuppressive effects of regulatory T cells, thereby limiting their secretion of IL-10 and TGF-β. This limiting of IL-10 and TGF-β secretion is correlated with better ADCC of the NK cells, validating the indirect stimulating effect of the anti-CD303 antibodies on the anticancer action of anti-AMHRII antibodies.

### 1.2- Effect of IL-10 and TGF-β on ADCC Activity of the Anti-AMHRII Antibody

To test the ADCC of the anti-AMHRII in a tumoral context involving either activation of pDCs or depletion of pDCs, the following protocol was set up:

NK effector cells transfected with CD16 («NK-CD16») were placed in culture at 3×10⁵ cells/ml in a culture medium containing IL-2.

At D-1, the NK-CD16 cells were placed in a culture medium without IL-2, in the absence or presence of IL-10 (5, 50 or 120 ng/ml) and TGF-β (5, 50 or 120 ng/ml).

At D0, COV434 cells (35000 cells/well) expressing AMHRII were incubated in a flat bottom 96-well plate with the NK-CD16 cells, in an E/T ratio (Effector-NK/Target-COV434) of 5:1 and 5000 ng/ml of anti-AMHRII antibody. After an incubation time of 16 hours at 37° C., the supernatant was collected.

The negative control followed the same protocol wherein the anti-AMHRII was replaced by a chimeric antibody inhibiting the anti-FVIII antibody, produced in YB2/0.

Lysis of the target cells induced by the anti-AMHRII antibodies was measured chromogenically by quantifying the intracellular lactate dehydrogenase enzyme (LDH) released into the supernatant by the lysed target cells (Roche Diagnostics—Cytotoxicity Detection Kit LDH).

Percent lysis was calculated with the following formula:

% lysis=[(ER−SR)/(100−SR)]−[(NC−SR)/(100−SR)]

Where ER and SR respectively represent experimental (ER9) and spontaneous (SR) release of LDH, and NC represents the natural cytotoxicity of NK cells. The results (% lysis) are expressed as a percentage, 100% being the value taken as reference obtained with the NK-CD16 cells in the presence of anti-AMHRII antibodies and in the absence of IL-10 and TGF-β (i.e. anti-AMHRII antibody alone).

Conclusion: Through cascade effect, comparison of percent lysis observable in the absence of IL-10 and/or TGF-β cytokines, compared with percent lysis observable in the presence of these same cytokines, allows evaluation of the impact of depleting pDCs present at the tumour site. It can thus be shown that anti-CD303 antibodies allow indirect potentializing of the effect of the anti-AMHRII anticancer antibodies.

### 1.3- Effect of IL-10 and TGF-G3 on Phagocytosis Induced by Anti-AMHRII

The monocytes were differentiated into CD16+microphages (M2 like) for 2 days in RPMI 1640 +10% SVF +M-CSF 50 ng/ml for 48 h.

The SKBR3 cells and macrophages were labelled with PKH-67 (green fluorescence) and PKH-26 (red fluorescence), respectively.

The SKBR3 cells were opsonized with 10 μg/ml of AMHRII antibody or with an irrelevant antibody and then incubated with the macrophages (1.105 of each cell/well) in the absence or presence of different concentrations of IL-10 (5 and 50 ng/ml) alone, of TGF-β (5 and 50 ng/ml) alone, and IL-10+TGF-β (5 and 50 ng/ml).

After incubation for 3 h at 37° C., the cells were placed in a counting chamber (Mallassez) and observed under a fluorescence microscope.

Percent phagocytosis was evaluated by counting the number of macrophages (at least 100 macrophages) containing COV434 cells.

Conclusion: Through cascade effect, comparison of percent phagocytosis observable in the absence of IL-10 and/or TGF-β cytokines, with percent lysis observable in the presence of these same cytokines, allows evaluation of the impact of depleting pDCs present at the tumour site. It is thus possible to show that the anti-CD303 antibodies allow indirect potentializing of the effect of the anti-AMHRII anticancer antibodies.

### Example 2 : Effect of an Anti-CD303 Antibody on Activation of Regulatory T Cells (Treg)

### 2.1-Role of Anti-CD303 on the Phenotype and Expansion of Tregs in PBMC

The mononuclear cells (PBMCs) were isolated from a tube of blood taken on anti-coagulant. The Treg cells were identified and subjected to phenotype characterization via flow cytometry on the basis of 3 markers: CD4, CD25 and Fox-P3.

Different quantities of anti-CD303 antibody (from 1 ng to 10 μg/ml) were added to the PBMCs in the presence of IL-2 (500 U/ml). The number of Tregs and their phenotype were monitored over time (1 to 4 days).

Under the same conditions, beads coated with anti-CD3/anti-CD28 to stimulate T proliferation were added in a Treg/beads ratio of 4:1 to verify Treg activation.

Conclusion: It can therefore be shown that the anti-CD303 antibodies, in the absence of pDCs, do not have any impact on the expansion and immunosuppressive phenotype of regulatory T cells.

### 2.2- Role of anti-CD303 on the Phenotype and Expansion of Purified Tregs in the Presence of pDCs

The Treg cells (CD⁴⁺, CD25⁺) were purified from PBMCs using a 2-step process: depletion of negative CD4 cells (positive cells for the CD8, CD14, CD15, CD16, CD19, CD36, CD56, CD123, TCRγ/δ and CD235a markers) followed by positive selection of CD25⁺cells.

Purified pDCs or pDC lines (e.g. obtained with the method described in Maeva CAL-1) were added in a Treg/pDC ratio of 100, 10 and 1. Different quantities of anti-CD303 antibody (from 1 ng to 10 μg/ml) were added to the Treg/pDC mixture in the presence of IL-2 (500 U/ml). The number of Tregs and their phenotype were monitored over time (1 to 4 days). Under the same conditions, beads coated with anti-CD3/anti-CD28 to stimulate T proliferation were added in a Treg/beads ratio of 4:1 to verify Treg activation.

A negative control in the absence of pDCs was prepared to verify the direct impact (expected to be neutral) of anti-CD303 on Tregs.

Conclusion : Observation over several days of the expansion and differentiation of purified

Tregs in the presence of pDCs, after administration of anti-CD303 antibody, can show that administration of anti-CD303 is effective in reducing or suppressing the immunosuppressive properties of pDCs.

### Example 3: Depletion of Human pDCs Via an Anti-CD303 Antibody in Vivo in the Treatment of Ovarian Cancer

### 3.1-Generation of a Reproductive Study Model of a Pathological Situation in Man

To study the effect of an anti-CD303 antibody in the treatment of ovarian cancer, a humanized tumour mouse model' (HTM) can be used. It is characterized by the development of a mature human immune system and the growth of human ovarian cancer cells previously co-transplanted with the human hemopoietic stem cells.

This model advantageously allows the grouping together of several relevant elements for reproducibility under in vivo conditions: presence of human pDCs which alone express the target CD303 on their surface, presence of infiltration by human Treg cells, presence of human tumour cells expressing AMHRII on their surface, molecule targeted by an anti-AMHRII and an immunocompetent murine host (effector cells of NK type for ADCC activity). The model previously described in the article by Wege et al. (Int. J. Cancer 2011: 129, 2194-2206) combines all these characteristics.

This model was adapted to make it compatible with BRGSF™ or BRGSF™A2 mice (BALB/c, Rag2tm1Fwa, IL-2Rγctm1Cgn, SIRPαNOD, Flk2tm1Irl, Tg(HLA-A/H2-D/B2M)1Bpe) characterized by the absence of T, B, and NK mouse cells, and solely expressing the HLA of human class 1, HLA-A2.1. Said BRGSFTM or BRGSFTM-A2 mice can be generated following the procedure described by Legrand N, Huntington N. Dak., Nagasawa M et al. (Functional CD47/signal regulatory protein alpha (SIRP(alpha)) interaction is required for optimal human T- and natural killer-(NK) cell homeostasis in vivo. Proc.Natl.Acad.Sci.U.S.A 2011;108:13224-13229). They acquire the genotype (HLA-A/H2-D/B2M)^{1Bpe} via transgenesis (Pascolo, S., N. Bervas, J. M. Ure, A. G. Smith, F. A. Lemonnier, and B. Perarnau. 1997. HLA-A2.1-restricted education and cytolytic activity of CD8(+) T lymphocytes from beta2 microglobulin (beta2m) HLA-A2.1 monochain transgenic H-2Db beta2m double knockout mice. J Exp Med 185:2043-2051).

### 3.2—Method Able to be Used to Test the Activity of the Anti-CD303 Antibody

In brief, new-born mice derived from the line of immunodeficient BRGSF-A2 mice (BALB/c Rag2^{tm1Fwa} IL-2Rγ_{c}^{tm1Cgn} SIRPα^{NOD} Flk2^{tm1Irl} Tg(HLA-A/H2-D/B2M)^{1Bpe} are irradiated (3 Gy) over the first 192 hours of their lifetime. Twenty-four hours later they are transplanted by intra-hepatic injection with 1.5×10⁵ human CD34+ cells isolated from umbilical cord blood (CB) in the presence or absence of 3×10⁶ COV434-AMHRII-Luc tumour cells (expressing luciferase for bioluminescent monitoring). The COV434 cells are cells of human origin derived from ovarian carcinoma expressing the AMHRII receptor on their surface, the target of the anti-AMHRII antibody. In this particular model, the COV.434 cells, before administration thereof, were modified by lentiviral transduction to make them luminescent through the constituent expression of luciferase. This modification provides transverse tracking of tumour penetration over time by means of bioluminescence analysis whilst not sacrificing the animals and thereby best adjusting the window for start of treatment. Eleven to twelve weeks after co-administration of human cells, the mice were tested for their extent of humanization by analysing the cell composition of their blood (human and murine) by flow cytometry, and divided into five different groups (cf. Table below: a control group without injection of COV434 cells, treated with an isotype antibody (i.e. an anti-Factor VIII inhibiting antibody) (this group was used as negative control for tumour penetration - Group 1); a control group with injection of COV434 cells, treated with an isotype antibody (i.e. an anti-Factor VIII inhibiting antibody) (Group 2); a group treated with transgenic anti-AMHRII (AATG)—Group 3); a group treated with the anti-CD303 antibody (Group 4); and a group treated with the combination of anti-CD303 antibody and AATG treatments (Group 5).

The doses administered and treatment frequencies are given in the Table below:

| | COV434- | | |
|---|---|---|---|
| Groups | AMHRII-Luc | Tested product(s) | Number |
| 1 | No | Isotype (anti-Factor VIII | n = 10 |
| | | inhibiting antibody) | |
| 2 | Yes | Isotype (anti-Factor VIII) | n = 10 |
| | | inhibiting antibody | |
| 3 | Yes | AATG | n = 10 |
| 4 | Yes | Anti-CD303 | n = 10 |
| 5 | Yes | AATG + anti-CD303 | n = 10 |

Treatment started 14 weeks after humanization (i.e. injection of human CD34+cells isolated from umbilical cord blood—CB) in the presence or absence of 3×10⁶ COV434-AMHRII-Luc tumour cells, and lasted 19 weeks.

For treatment, the tested products were injected via intravenous route at a dose of 30 mg/kg bodyweight for the anti-CD303 antibody every 3 days, and once a week at 10 mg/kg bodyweight for the AATG antibody. The bodyweight of the mice was determined 3 days before the start of treatment for individual dose adjustment.

Blood samples were frequently taken to test the efficacy of human pDC depletion, by flow cytometry. In addition, bioluminescent analysis at the start of treatment and subsequently every two weeks was performed to compare the efficacy of the different tested products. Finally, tumour analysis in three animals per group at week 18 after humanization was carried out by flow cytometry to verify the presence or absence of human pDCs infiltrating the tumours.

### Results

The impact of the treatment on the human pDC sub-population, and the other populations of lymphoid cells (B lymphocytes B, T lymphocytes . . . ) present in the blood and spleen, was determined by flow cytometry at different times: D1, D3 and D7. The results show that treatment with the anti-CD303 antibody at a dose of 30 mg/kg in humanized BRGSF-HIS mice induces depletion of human pDCs for at least 7 days in the blood and spleen. In the blood, the depleting activity on human pDCs was rapid (>80% on Day 1) but occurred later in the spleen. In the blood and spleen, depletion of human pDCs was always efficient (>90%) on Day 7, i.e. 3 days after the last injection of the monoclonal anti-CD303 antibody. It is to be noted that the depleting activity of the anti-CD303 antibody was highly specific since it did not significantly affect the other sub-populations of human hemopoietic cells in the tested organs.

Additionally, it is to be noted that this model proves to be a particularly suitable model for the study of the invention, since the detecting of pDCs in the tumours of sacrificed mice can show the presence of pDCs infiltrating these tumours.

### Conclusion:

The adapted HTM mouse model can advantageously be used to evaluate the indirect impact of anti-CD303 antibody administration on the effect of the anti-ovarian tumour agent: the anti-AMHRII antibody, under conditions reproducing a physiological situation in vivo, in particular by comparing the results obtained with the different tested groups.

This model is therefore useful in evaluating the benefit, and advantageously the synergic effect, of administering an anti-CD303 antibody in combination with administration of an anti-AMHRII antibody in an ovarian tumour.

1. Pharmaceutical composition comprising, in a pharmaceutically acceptable carrier:
a. at least one anti-CD303 antibody; and
b. at least one anti-AMHRII antibody.

2. A method for preventing or treating an urogenital cancer in a subject in need thereof, comprising administering to said subject products containing:
a. at least one anti-CD303 antibody; and
b. at least one anti-AMHRII antibody,
which are combination products for simultaneous, separate or sequential.

3. Pharmaceutical composition according to claim 1, wherein the antibodies a) and b) are selected from among mouse antibodies, chimeric antibodies, humanized antibodies and human antibodies.

4. Pharmaceutical composition according to claim 1, wherein the anti-CD303 antibody is a monoclonal antibody directed against the ectodomain of the human CD303 antigen.

5. Pharmaceutical composition according to claim 1, wherein the anti-CD303 antibody comprises the following CDRs:

| | | |
|---|---|---|
| CDR1-H-IMGT-122A2 | GYTFTDYS | (SEQ ID NO: 13) |
| CDR2-H-IMGT-122A2 | ISTYYGDS | (SEQ ID NO: 14) |
| CDR3-H-IMGT-122A2 | ARNGNFYVMDY | (SEQ ID NO: 15) |
| CDR1-L-IMGT-122A2 | QDISNY | (SEQ ID NO: 16) |
| CDR2-L-IMGT-122A2 | YTS | (SEQ ID NO: 17) |
| CDR3-L-IMGT-122A2 | QQGNTLPWT | (SEQ ID NO: 18) |

or else:

| | | |
|---|---|---|
| CDR1-H-IMGT-102E9 | GYTFTDYS | (SEQ ID NO: 19) |
| CDR2-H-IMGT-102E9 | INTETGEP | (SEQ ID NO: 20) |
| CDR3-H-IMGT-102E9 | TRNGYYVGYYAMDY | (SEQ ID NO: 21) |
| CDR1-L-IMGT-102E9 | SSVIY | (SEQ ID NO: 22) |
| CDR2-L-IMGT-102E9 | STS | (SEQ ID NO: 23) |
| CDR3-L-IMGT-102E9 | QQRRSYPFT | (SEQ ID NO: 24) |

or else:

| | | |
|---|---|---|
| CDR1-H-IMGT-104C12 | GYTFTDYS | (SEQ ID NO: 25) |
| CDR2-H-IMGT-104C12 | ISPYYGDT | (SEQ ID NO: 26) |
| CDR3-H-IMGT-104C12 | ARNDDYYRFAY | (SEQ ID NO: 27) |
| CDR1-L-IMGT-104C12 | QDINNY | (SEQ ID NO: 28) |
| CDR2-L-IMGT-104C12 | YTS | (SEQ ID NO: 29) |
| CDR3-L-IMGT-104C12 | QQGKTLPWT | (SEQ ID NO: 30) |

or else:

| | | |
|---|---|---|
| CDR1-H-IMGT-114D11 | GYTFTDSS | (SEQ ID NO: 31) |
| CDR2-H-IMGT-114D11 | INTETGGP | (SEQ ID NO: 32) |
| CDR3-H-IMGT-114D11 | ARNGYYVGYYALDY | (SEQ ID NO: 33) |
| CDR1-L-IMGT-114D11 | SSVFY | (SEQ ID NO: 34) |
| CDR2-L-IMGT-114D11 | STS | (SEQ ID NO: 35) |
| CDR3-L-IMGT-114D11 | QQRRSYPYT | (SEQ ID NO: 36) |

or else:

| | | |
|---|---|---|
| CDR1-H-IMGT-104E10 | GYTFTDYS | (SEQ ID NO: 37) |
| CDR2-H-IMGT-104E10 | INTETGEP | (SEQ ID NO: 38) |
| CDR3-H-IMGT-104E10 | ARNGYYVGYYAMDY | (SEQ ID NO: 39) |
| CDR1-L-IMGT-104E10 | SSVIY | (SEQ ID NO: 40) |
| CDR2-L-IMGT-104E10 | STS | (SEQ ID NO: 41) |
| CDR3-L-IMGT-104E10 | QQRRSYPYT | (SEQ ID NO: 42) |

6. Pharmaceutical composition according to claim 1, wherein the anti-AMRHII antibody comprises:
a) a light chain comprising or composed of a variable region in which the amino acid sequence is represented by:
SEQ ID NO: 65, or
SEQ ID NO: 66, or
SEQ ID NO: 67, or
SEQ ID NO: 68,
and of a constant region in which the amino acid sequence is represented by SEQ ID NO: 69;
b) a heavy chain comprising or composed of a variable region in which the amino acid sequence is represented by:
SEQ ID NO: 70,
SEQ ID NO: 71,
SEQ ID NO: 72,
SEQ ID NO: 73,
SEQ ID NO: 74,
and of a constant region in which the amino acid sequence is represented by SEQ ID NO: 75.

7. Pharmaceutical composition according to claim 1, wherein the anti-AMRHII antibody comprises:
a) a light chain composed of the amino acid sequence represented by SEQ ID NO: 76, and b) a heavy chain composed of the amino acid sequence represented by SEQ ID NO: 77, or, a) a light chain composed of the amino acid sequence represented by SEQ ID NO: 78, and b) a heavy chain composed of the amino acid sequence represented by SEQ ID NO: 79, or, a) a light chain composed of the amino acid sequence represented by SEQ ID NO: 80, and b) a heavy chain composed of the amino acid sequence represented by SEQ ID NO: 81, or, a) a light chain composed of the amino acid sequence represented by SEQ ID NO:82, and b) a heavy chain composed of the amino acid sequence represented by SEQ ID NO: 83, or, a) a light chain composed of the amino acid sequence represented by SEQ ID NO: 84, and b) a heavy chain composed of the amino acid sequence represented by SEQ ID NO: 85.

8. Pharmaceutical composition according to claim 1, wherein the anti-AMRHII antibody comprises three heavy chain CDRs having the following amino acid sequences or sequences having at least 80% identity with the following sequences, and three light chain CDRs having the following amino acid sequences or sequences having at least 80% identity with the following sequences:
CDR1-H 3C_23: GYTFTSHY (SEQ ID NO: 87),
CDR2-H 3C_23: IYPGDDST (SEQ ID NO: 88),
CDR3-H 3C_23: TRGDRFAY (SEQ ID NO: 89),
CDR1-L 3C_23: SSVRY (SEQ ID NO: 90),
CDR2-L 3C_23: PTS and
CDR3-L 3C_23: LQWSSYPWT (SEQ ID NO: 91).

9. Pharmaceutical composition according to claim 1, wherein the anti-CD303 antibody and/or anti-AMHRII antibody is selected from among Fab, F(ab)′f2, Fd, scFv and multimers of scFv.

10. A method for preventing or treating a urogenital cancer in a subject in need thereof, comprising administering to said subject a Pharmaceutical composition according to claim 1.

11. The method according to claim 10, wherein the urogenital cancer is ovarian cancer.

12. The method according to claim 2, wherein the antibodies a) and b) are selected from among mouse antibodies, chimeric antibodies, humanized antibodies and human antibodies.

13. The method according to claim 2, wherein the anti-CD303 antibody is a monoclonal antibody directed against the ectodomain of the human CD303 antigen.

14. The method according to claim 2, wherein the anti-CD303 antibody comprises the following CDRs:

| | | |
|---|---|---|
| CDR1-H-IMGT-122A2 | GYTFTDYS | (SEQ ID NO: 13) |
| CDR2-H-IMGT-122A2 | ISTYYGDS | (SEQ ID NO: 14) |
| CDR3-H-IMGT-122A2 | ARNGNFYVMDY | (SEQ ID NO: 15) |
| CDR1-L-IMGT-122A2 | QDISNY | (SEQ ID NO: 16) |
| CDR2-L-IMGT-122A2 | YTS | (SEQ ID NO: 17) |
| CDR3-L-IMGT-122A2 | QQGNTLPWT | (SEQ ID NO: 18) |

or else:

| | | |
|---|---|---|
| CDR1-H-IMGT-102E9 | GYTFTDYS | (SEQ ID NO: 19) |
| CDR2-H-IMGT-102E9 | INTETGEP | (SEQ ID NO: 20) |
| CDR3-H-IMGT-102E9 | TRNGYYVGYYAMDY | (SEQ ID NO: 21) |
| CDR1-L-IMGT-102E9 | SSVIY | (SEQ ID NO: 22) |
| CDR2-L-IMGT-102E9 | STS | (SEQ ID NO: 23) |
| CDR3-L-IMGT-102E9 | QQRRSYPFT | (SEQ ID NO: 24) |

or else:

| | | |
|---|---|---|
| CDR1-H-IMGT-104C12 | GYTFTDYS | (SEQ ID NO: 25) |
| CDR2-H-IMGT-104C12 | ISPYYGDT | (SEQ ID NO: 26) |
| CDR3-H-IMGT-104C12 | ARNDDYYRFAY | (SEQ ID NO: 27) |
| CDR1-L-IMGT-104C12 | QDINNY | (SEQ ID NO: 28) |
| CDR2-L-IMGT-104C12 | YTS | (SEQ ID NO: 29) |
| CDR3-L-IMGT-104C12 | QQGKTLPWT | (SEQ ID NO: 30) |

or else:

| | | |
|---|---|---|
| CDR1-H-IMGT-114D11 | GYTFTDSS | (SEQ ID NO: 31) |
| CDR2-H-IMGT-114D11 | INTETGGP | (SEQ ID NO: 32) |
| CDR3-H-IMGT-114D11 | ARNGYYVGYYALDY | (SEQ ID NO: 33) |
| CDR1-L-IMGT-114D11 | SSVFY | (SEQ ID NO: 34) |
| CDR2-L-IMGT-114D11 | STS | (SEQ ID NO: 35) |
| CDR3-L-IMGT-114D11 | QQRRSYPYT | (SEQ ID NO: 36) |

or else:

| | | |
|---|---|---|
| CDR1-H-IMGT-104E10 | GYTFTDYS | (SEQ ID NO: 37) |
| CDR2-H-IMGT-104E10 | INTETGEP | (SEQ ID NO: 38) |
| CDR3-H-IMGT-104E10 | ARNGYYVGYYAMDY | (SEQ ID NO: 39) |
| CDR1-L-IMGT-104E10 | SSVIY | (SEQ ID NO: 40) |
| CDR2-L-IMGT-104E10 | STS | (SEQ ID NO: 41) |
| CDR3-L-IMGT-104E10 | QQRRSYPYT | (SEQ ID NO: 42) |

15. The method according to claim 2, wherein the anti-AMRHII antibody comprises:
a) a light chain comprising or composed of a variable region in which the amino acid sequence is represented by:
SEQ ID NO: 65, or
SEQ ID NO: 66, or
SEQ ID NO: 67, or
SEQ ID NO: 68,
and of a constant region in which the amino acid sequence is represented by SEQ ID NO: 69;
b) a heavy chain comprising or composed of a variable region in which the amino acid sequence is represented by:
SEQ ID NO: 70,
SEQ ID NO: 71,
SEQ ID NO: 72,
SEQ ID NO: 73,
SEQ ID NO: 74,
and of a constant region in which the amino acid sequence is represented by SEQ ID NO: 75.

16. The method according to claim 2, wherein the anti-AMRHII antibody comprises:
a) a light chain composed of the amino acid sequence represented by SEQ ID NO: 76, and
b) a heavy chain composed of the amino acid sequence represented by SEQ ID NO: 77, or,
a) a light chain composed of the amino acid sequence represented by SEQ ID NO: 78, and
b) a heavy chain composed of the amino acid sequence represented by SEQ ID NO: 79, or,
a) a light chain composed of the amino acid sequence represented by SEQ ID NO: 80, and
b) a heavy chain composed of the amino acid sequence represented by SEQ ID NO: 81, or,
a) a light chain composed of the amino acid sequence represented by SEQ ID NO:82, and
b) a heavy chain composed of the amino acid sequence represented by SEQ ID NO: 83, or,
a) a light chain composed of the amino acid sequence represented by SEQ ID NO: 84, and
b) a heavy chain composed of the amino acid sequence represented by SEQ ID NO: 85.

17. The method according to claim 2, wherein the anti-AMRHII antibody comprises three heavy chain CDRs having the following amino acid sequences or sequences having at least 80% identity with the following sequences, and three light chain CDRs having the following amino acid sequences or sequences having at least 80% identity with the following sequences:
CDR1-H 3C_23: GYTFTSHY (SEQ ID NO: 87),
CDR2-H 3C_23: IYPGDDST (SEQ ID NO: 88),
CDR3-H 3C_23: TRGDRFAY (SEQ ID NO: 89),
CDR1-L 3C_23: SSVRY (SEQ ID NO: 90),
CDR2-L 3C 23: PTS and
CDR3-L 3C_23: LQWSSYPWT (SEQ ID NO: 91).

18. The method according to claim 2, wherein the anti-CD303 antibody and/or anti-AMHRII antibody is selected from among Fab, F(ab)′2, Fd, scFv and multimers of scFv.

19. The method according to claim 2, wherein the urogenital cancer is ovarian cancer.

20. Pharmaceutical composition according to claim 3, wherein the anti-CD303 antibody is a monoclonal antibody directed against the ectodomain of the human CD303 antigen.
